(19) United States
(12) Patent Application Publication
(10) Pub. No.: US 2012/0215098 A1
(43) Pub. Date: Aug. 23, 2012

(54) Trans-Catheter / Trans-Endoscope Drug and Stem Cell Delivery

(76) Inventors: John F Black, San Mateo, CA (US); Jayakumar Rajadas, Cupertino, CA (US); Geoffrey Gurtner, Stanford, CA (US)

(21) Appl. No.: 13/402,562

(22) Filed: Feb. 22, 2012

Related U.S. Application Data
(60) Provisional application No. 61/463,811, filed on Feb. 22, 2011.

(51) Int. Cl. A61B 6/00; A61B 1/04; A61M 5/00

(52) U.S. Cl. 600/431; 604/131; 600/109

(57) ABSTRACT

An apparatus and method are provided for very precise and efficient delivery of e.g. viscous nutritive cell matrices and/or drugs into an exact point in the human body using minimally-invasive surgical techniques. Embodiments are compatible with modern catheter access and endoscopic techniques and a disposable-plus-capital-equipment business model separating the cost of the procedure between a reusable and a disposable component. It also represents a substantial step forward in terms of safety with no high voltage or high pressure components present in the body. The inherent risk of using this design to deliver substances into the human body is significantly reduced compared to standard hydraulic methods. Mechanical trauma associated with needles is avoided with this invention, and the method is also compatible with tortuous anatomy such as the coronary or brain arteries.

### CROSS-REFERENCE TO RELATED APPLICATIONS

This application claims priority from U.S. Provisional Patent Application 61/463,811 filed Feb. 22, 2011, which is incorporated herein by reference.

### FIELD OF THE INVENTION

The invention relates to trans-catheter or trans-endoscope drug- and stem cell delivery devices and methods.

### BACKGROUND OF THE INVENTION

There is a need to provide drug or stem-cell delivery systems compatible with the small internal diameters of the coronary, cranial or peripheral vasculature. Likewise there is a need to provide drug delivery systems that are compatible with the small working diameters of modern endoscopes, laparoscopes and other minimally invasive surgical tools. For catheters designed for delivery of for example drugs in the internal vasculature, one would generally prefer to avoid high pressure gas and/or liquid transmission lines being present in the catheter, as failure of one of these lines might cause a dissection, perforation or embolism. One would generally like to avoid the use of high voltage carrying wires in the catheters also for safety reasons. The use of mechanical linkages, either rotational or moving longitudinally suffers from loss of mechanical energy delivered as a result of the effects of the static and dynamic coefficients of friction between the elements in the catheters. For both endoscopic and catheter delivery there is a general need to provide injection systems that can follow tortuous anatomy without breaking or without perforating the tissue, placing an emphasis on the use of flexible materials and small crossing profiles. Accordingly, there is a need in the art to allow drugs or stem cells to be delivered precisely to various body tissues using a catheter or endoscope in a manner that is intrinsically safer than available previously, compatible with tortuous anatomy and consistent with a low cost disposable business model.

### SUMMARY OF THE INVENTION

An apparatus for delivering substances or compositions to a target (e.g. biological tissue) is provided. The apparatus is either part of or is a catheter or an endoscope with an outer diameter on the order of less or equal to 2 mm. In one aspect, the outer diameter is suitable to gain access to or move within a coronary vasculature, a peripheral vasculature, or a cranial vasculature.

A housing distinguishes a first chamber holding a deliverable material near its distal end and a second chamber holding a material capable of a phase transition near its proximal end. The housing has one or more openings at its distal end for delivery of the deliverable material. The first chamber and the second chamber are separated by a partition or movable membrane, which is movable within the housing.

A fiber optic light guide is positioned near the proximal end, whereby the optically guiding portion of the fiber optic light guide is in optical contact with the phase transitionable material, allowing optical energy to pass substantially unimpeded from the fiber light guide to the second chamber housing the phase transitionable material. The material capable of a phase transition exhibits a solid-to-vapor phase transition or a liquid-to-vapor phase transition in response to interaction with the optical energy.

A volume-expansion phase transition of the phase-transitionable material is induced by absorption of the optical energy delivered by the fiber optic light guide. This induced volume-expansion phase-transition causes movement of the partition in a proximal to distal direction in the housing which causes ejection of the deliverable material through one or more of the openings at the distal end of the housing. The expansion chamber could have one or more vents for venting vapor from the second chamber either into another chamber or lumen.

In one aspect, the optical energy is sufficient to cause a volume-expansion to be in the order 100-500 times of the original volume of the material in the second chamber. In another aspect, the optical energy is sufficient to cause a volume-expansion to be in the order 100 times of the original volume of the material in the second chamber. In yet another aspect, the optical energy is sufficient to cause a volume-expansion to be in the order 50 times of the original volume of the material in the second chamber.

### BRIEF DESCRIPTION OF THE DRAWING

FIGS. 1-2 show the basic design apparatus according to an exemplary embodiment of the invention. The imaging device 190 can be integrated with the catheter or endoscope or used in conjunction with a catheter or an endoscope (i.e. this allows for both externally and internally image guided devices). As shown in FIG. 2, the apparatus could be modified for side firing, for example, to deliver a deliverable material to the walls of an artery or vein, by placing one or more holes 114 to eject the material on the sides of chamber 1 (120) instead of at the distal tip 112.

### DETAILED DESCRIPTION

Embodiments described herein pertain to a device 100 with a distal tip 112 having a two chambered assembly, respectively chamber 1 (120) and chamber 2 (130).

Device 100 could be a catheter or endoscope or could be part of a catheter or endoscope. Chamber 2 (130) is optically contacted to a fiber optic light guide 150. In one embodiment, chamber 2 (130) and the fiber optic light guide 150 are separated by a window 160, which is optically transparent to the light coming through fiber optic light guide 150. The fiber optic light guide leads back to a laser 170 and could have one or more optical fibers capable of transmitting at, for example, a wavelength of about 2940 nm.

Chamber 1 (120) is distal of chamber 2 (130) and acts as a reservoir for a deliverable material. Examples of deliverable materials are, without any limitations, stem cells, one or more drugs, chemotherapeutics, one or more hydrogel support matrices, tissue adhesives, tissue soldering compositions, radioactive brachytherapy substances, imaging contrast agents (Xray, MRI, CT, Optical, Ultrasound), adhesion-preventing agents, tissue-lubricating agents (e.g. hyaluronic acid, or the like), marking compounds and fiducial markers to enable and guide surgery, biodegradable drug eluting compounds or compositions, inert matrices or scaffolds to promote bone growth or melding.

Chamber 2 (130) is filled with a material that is capable of a liquid-to-vapor or solid-to-vapor phase transition, and which can absorb the laser radiation strongly, defined as having an absorption coefficient such that at least (1−e⁻³) of the radiation is absorbed. Examples of materials capable of such phase transition, are without any limitations, water, isotonic saline, a biocompatible isotonic liquid, a low-boiling-point biocompatible liquid, a low-sublimation-point biocompatible solid, a hydrocarbon-based wax, a hydrocarbon-based liquid, or sulfur hexafluoride. In general, any biocompatible material exhibiting a solid-to-vapor or liquid-to-vapor phase transition in response to heating (from the optical energy) may be used.

The two chambers in the catheter are separated by a movable membrane 140 or other type of moving partition. Movable membrane or partition 140 is made of a material having a thermal conductivity and a thermal expansion coefficient such that heat resulting as a by-product from the phase transition is not transferred rapidly to the injectable material and surrounding tissue, and such that the movable partition has a low risk of jamming in said housing during said volume-expansion.

When the laser is triggered, a laser pulse is (or pulses are) sent down the optical fiber guide from a proximal to distal direction. When the laser pulse impinges (or pulses impinge) on the material in chamber 2 (130), the optical energy is strongly absorbed by the material such that at least (1−e⁻³) of the radiation is absorbed and the material is converted to gas on a timescale of tens of microseconds to hundreds of milliseconds which is accompanied by a large fast volume expansion and pressure increase. In one example, the volume expansion caused by the fiber optic of the phase-transitionable material is in the order of 100-500 times of its original volume. In another example, the expansion is at least 50 times, and in yet another example the expansion is at least 100 times. The volume expansion can be titrated vs. external back-pressure using the laser pulse energy and wavelength as this determines how much of the phase transition material changes to the gas phase and the absorption depth in the phase-transitionable material.

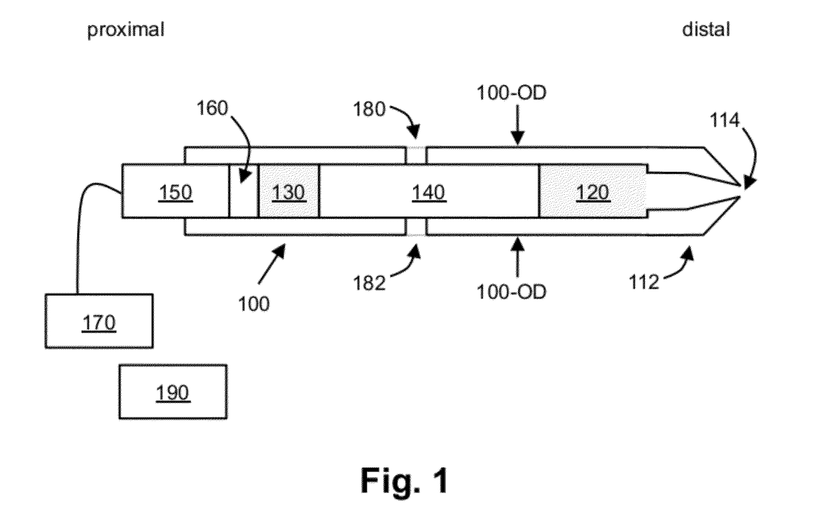

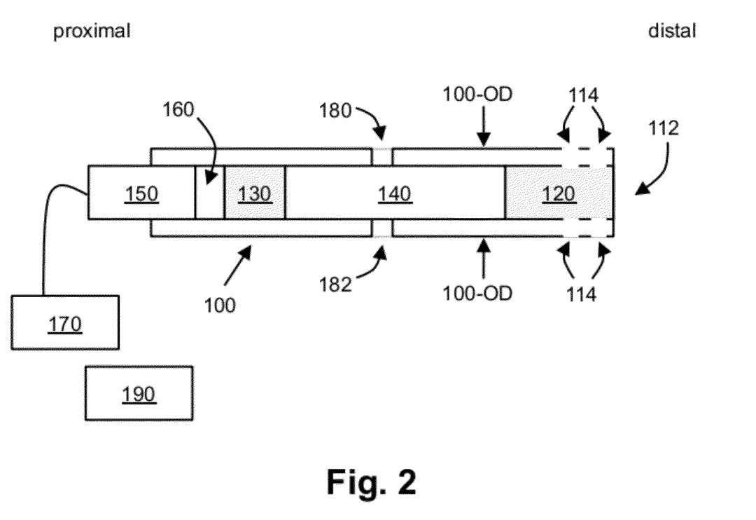

This pressure increase (volume expansion) pushes on movable membrane or partition 140 between chamber 1 (120) and chamber 2 (130), forcing the contents of chamber 1 (120) through one or more opening(s) 114 (e.g. a deLaval nozzle in FIG. 1), which has been placed in contact with the tissue receiving the deliverable material. The expansion of the volume of chamber 2 (130) is sufficiently fast (tens of microseconds to hundreds of milliseconds) that a very large pressure differential on the order of tens of atmospheres may be achieved, forcing the contents of chamber 1 (120) into the tissue (not shown). The device may be equipped with an imaging capability or device 190 to facilitate image guided placement of the contents of chamber 1 (120), for example specifically to the site of a myocardial infarction or ischemic stroke. The imaging device 190 can be integrated with the catheter or endoscope or used in conjunction with a catheter or an endoscope (i.e. this allows for both externally and internally image guided devices. Imaging could be for example intravascular ultrasound or intravascular OCT or an optical imaging bundle.

In one embodiment, the second (expansion) chamber contains one or more vents 180, 182 for venting vapor from the second chamber into a volume (not shown) containing isotonic saline or water such that the phase transition is quenched once the partition has moved such that (i) the expansion is arrested safely and without hot gas being discharged into the surrounding tissue and (ii) the phase transitionable material is returned to its pre-expansion state without an accompanying recoil of the partition.

In another embodiment, second (expansion) chamber could also contain one or more vents 180, 182 for venting vapor from the second (expansion) chamber into a third lumen (not shown) on a catheter or an endoscope such that (i) the vapor generated in the phase transition is allowed to expand into the lumen such that the expansion is arrested safely and without hot gas being discharged into the surrounding tissue and (ii) the phase transitionable material is returned to its pre-expansion state without an accompanying recoil of the partition.

The choice of laser wavelength is linked to the choice of the phase transition material in chamber 2 (130) of the device. Once the phase transition material has been chosen, any suitable laser emitting at wavelengths strongly absorbed by the material can be used and connected to the fiber optic. The absorption criterion is having almost complete absorption (i.e. at least (1−e⁻³)) of the delivered laser energy in a distance significantly shorter than the physical length of the phase transition material in chamber 2 (130) so that little or no unabsorbed laser energy hits the partition or other components of the catheter or endoscope. In the example of water or isotonic saline, the fiber optical light guide can be connected to an Erbium:YAG laser emitting light at about 2.94 microns, a CTH:YAG (“Holmium:YAG)”) laser emitting at about 2.08 microns, a carbon dioxide laser emitting at about 10.6 microns, or a carbon monoxide laser emitting at about 5 microns. In another example of water or isotonic saline, the fiber optical light guide can be connected to an optical parametric oscillator emitting light at around 3 microns or emitting at around 1.9-2.1 microns.

The wavelength can be tuned in a discrete manner to alter the absorption depth in the water and hence the properties of the expansion. For example, the peak absorption is at 3 microns which coincides nicely with an Er:YAG laser at 2.94 microns. The absorption depth here (e⁻³) is on the order of 10 microns which will give a very quick absorption and expansion. Selection of other wavelengths could be used to perform slower (hundreds of milliseconds to seconds) injections with very large back pressure where rapid injection of the substance was not required or optimal. It is likely that any wavelength that is strongly/completely absorbed in around 100-200 microns of water will be viable for this technique, so almost any wavelength longer than 2-5-2.6 microns and also the peak at 1.9 microns. If the absorption depth is too long, however, there is a risk that the energy will start to migrate to the surrounding tissue giving collateral damage. Suitable lasers in this range are erbium and holmium YAG (CTH), carbon dioxide, carbon monoxide, OPOs, possibly thulium fiber lasers at 1.9 microns, erbium:YSGG at 2.76 microns.

Additional materials could be constructed by dissolving an absorber in another material, for example a solution of indocyanine green (ICG—an FDA approved dye) in water could be used and where the chromophore for absorption is now the ICG, absorbing laser energy around 800 nm. The reason to consider an approach like this is that in general visible/near-IR wavelengths out to around 1.8 microns are easier to deliver using fiber optics than their counterparts at 2-10 microns.

The diameter 100-OD of the device or housing 100 is less or equal than 2 mm. In general, the diameter of the device can be determined by either the sheath size used to gain access to a coronary, a peripheral or a cranial vasculature (e.g. 5-6 French for coronary, 6-8 French for peripheral vasculature, 4-6 Fr for cranial or the utility channel size on the endoscope. In general, a good target OD is <2 mm for a device for vascular work. In addition, the rigid length of any part of the device should be minimized to allow the device to cross tortuous anatomy without breaking and without creating a perforation or dissection.

The openings at the distal tip 112 (i.e. nozzle) of the housing 100 can be one or multiple. The direction of the openings 114 can also be in any direction relative to the housing tip (e.g. in line with the longitudinal direction of the housing, to the sides of that axis, or under an angle relative to the longitudinal axis on the housing).

Embodiments of the invention can be used anywhere there is a need to inject a deliverable material precisely and where use of more traditional methods, for example, a syringe or power injector or other mechanical device is precluded or unfavorable. For example, injection of substances locally and precisely during a minimally invasive procedure (endoscope, laparoscope, catheter, NOTES (Natural Orifice Transluminal Endoscopic Surgery) etc). In addition, embodiments of the invention are useful anywhere one would like to minimize mechanical trauma to the injection site, or be very precise in depth or position.

Embodiments of the invention can be applied to trans-catheter delivery of drugs to the heart, coronary arteries, brain tissue, tumor sites etc. Image guided delivery of therapeutic stem cells in a matrix to damaged tissue, for example sites of myocardial infarction, sites of laser trans-myocardial revascularization or ischemic stroke. Trans-catheter or trans-endoscope delivery of water-sensitive systems, for example hydrogel matrices.

Advantages of embodiments of the invention are for example that such a device delivers the energy to the distal tip of the catheter using fiber optics which have very small impact on the crossing profile of the catheter, making it suitable for use in tortuous anatomy such as in the coronary arteries or arteries of the brain. Fiber optics are intrinsically very efficient at delivering energy as opposed to for example torque shafts which suffer from significant losses due to static and dynamic friction, and hysteresis (“wind-up” or “snaking”) losses. The pressure differential generated by the laser-initiated ‘bubble’ can in principal be very large, allowing the device to overcome local resistance to injection in even very dense or fibrotic tissue. The pressure differential can also be made sufficiently large that we may be able to inject very viscous substances, for example hydrogel support matrices, the viscosity of which might render syringe or other mechanically actuated injection schemes too difficult, particularly at the distal tip of a small catheter. Embodiments of the invention avoid having a high pressure gas or saline line connected to the patient which would be implicitly dangerous in the event of a valve failure. In addition, embodiments of the invention do not involve inserting a needle deep into the tissue, reducing trauma to the injection site and simplifying the design of the device. Furthermore the method of using a device according to an embodiment of the invention is very efficient at delivering small quantities of expensive reagents, for example stem cells in a matrix as it does not have a large dead volume (the whole length of the catheter for example). Use of the device is very well suited to a low cost-of-goods disposable device as there is no complicated proximal mechanism included in the disposable portion. A fiber optic connects the disposable catheter to the capital equipment portion of the system, separating the expensive parts of the assembly to the reusable section.

What is claimed is:

1. An apparatus for delivering substances or compositions to a target, comprising:
(a) a housing with a proximal end and a distal end, wherein said distal end comprises at least one opening, said housing distinguishing a first chamber near the distal end holding a deliverable material, and a second chamber near the proximal end to hold a material capable of a phase transition, wherein said first chamber and said second chamber are separated by a partition, wherein said partition is movable within said housing; and
(b) a fiber optic light guide, wherein the optically guiding portion of said fiber optic light guide is in optical contact with said phase transitionable material, allowing optical energy to pass substantially unimpeded from said optically guiding portion to said second chamber housing said phase transitionable material,
wherein a volume-expansion phase transition of said phase-transitionable material is induced by absorption of the optical energy delivered by said fiber optic light guide, wherein said induced volume-expansion phase-transition causes movement of said partition in a proximal to distal direction in said housing, and wherein said movement of said partition causes ejection of said deliverable material though said at least one opening.

2. The apparatus as set forth in claim 1, wherein said target is biological tissue.

3. The apparatus as set forth in claim 1, wherein said deliverable material comprises stem cells, one or more drugs, chemotherapeutics, one or more hydrogel support matrices, tissue adhesives, tissue soldering compositions, radioactive brachytherapy substances, imaging contrast agents, adhesion-preventing agents, tissue-lubricating agents, marking compounds and fiducial markers to enable and guide a surgery, biodegradable drug eluting compounds or compositions, or inert matrices or scaffolds to promote bone growth or melding.

4. The apparatus as set forth in claim 1, wherein said material capable of said phase transition exhibits a solid-to-vapor phase transition or a liquid-to-vapor phase transition in response to heating from said optical energy.

5. The apparatus as set forth in claim 1, wherein said material capable of said phase transition comprises water, isotonic saline, a biocompatible isotonic liquid, a low-boiling-point biocompatible liquid, a low-sublimation-point biocompatible solid, a hydrocarbon-based wax, a hydrocarbon-based liquid, or sulfur hexafluoride.

6. The apparatus as set forth in claim 1, wherein said optical energy is sufficient to cause said volume-expansion to be in the order 100-500 times of the original volume of said material in said second chamber.

7. The apparatus as set forth in claim 1, wherein said optical energy is sufficient to cause said volume-expansion to be in the order 100 times of the original volume of said material in said second chamber.

8. The apparatus as set forth in claim 1, wherein said optical energy is sufficient to cause said volume-expansion to be in the order 50 times of the original volume of said material in said second chamber.

9. The apparatus as set forth in claim 1, wherein said fiber optical light guide is connected to a source of pulsed infrared radiation at a wavelength that is strongly absorbed by said phase transitionable material.

10. The apparatus as set forth in claim 1, wherein said phase transitionable material is water or isotonic saline and wherein said fiber optical light guide is connected to an Erbium:YAG laser emitting light at about 2.94 microns, a CTH:YAG laser emitting at about 2.08 microns, a carbon dioxide laser emitting at about 10.6 microns, or a carbon monoxide laser emitting at about 5 microns.

11. The apparatus as set forth in claim 1, wherein said phase transitionable material is water or isotonic saline and wherein said fiber optical light guide is connected at the proximal end to an optical parametric oscillator emitting light at around 3 microns or emitting at around 1.9-2.1 microns.

12. The apparatus as set forth in claim 1, wherein said fiber optical light guide comprises one or more optical fibers capable of transmitting at a wavelength of about 2940 nm.

13. The apparatus as set forth in claim 1, wherein said partition is made of a material having a thermal conductivity and a thermal expansion coefficient such that heat resulting as a by-product from the phase transition is not transferred rapidly to the injectable material and surrounding tissue, and such that the movable partition has a low risk of jamming in said housing during said volume-expansion.

14. The apparatus as set forth in claim 1, wherein said expansion chamber contains one or more vents for venting vapor from said second chamber.

15. The apparatus as set forth in claim 1, wherein said apparatus is part of or is a catheter or an endoscope.

16. The apparatus as set forth in claim 1, wherein said apparatus has an outer diameter in the order of less or equal to 2 mm.

17. The apparatus as set forth in claim 1, wherein said apparatus has an outer diameter suitable to gain access to or move within a coronary vasculature, a peripheral vasculature, or a cranial vasculature.

18. The apparatus as set forth in claim 1, wherein said apparatus incorporates an imaging device.
